(19) United States
(12) Patent Application Publication
(10) Pub. No.: US 2018/0126037 A1
(43) Pub. Date: May 10, 2018

(54) DECELLULARIZED ORGAN-DERIVED TISSUE ENGINEERING SCAFFOLDS

(71) Applicants: THE TRUSTEES OF THE UNIVERSITY OF PENNSYLVANIA, Philadelphia, PA (US); POHANG UNIVERSITY OF SCIENCE AND TECHNOLOGY, Gyeongbuk (KR)

(72) Inventors: Dongeun Huh, Villanova, PA (US); Ju Young Park, Kyungbuk (KR); Cassidy Blundell, Philadelphia, PA (US); Dong-Woo Cho, Seoul (KR)

(73) Assignees: THE TRUSTEES OF THE UNIVERSITY OF PENNSYLVANIA, Philadelphia, PA (US); POHANG UNIVERSITY OF SCIENCE AND TECHNOLOGY, Gyeongbuk (KR)

(21) Appl. No.: 15/863,513

(22) Filed: Jan. 5, 2018

Related U.S. Application Data
(63) Continuation of application No. PCT/US2016/041566, filed on Jul. 8, 2016.
(60) Provisional application No. 62/190,130, filed on Jul. 8, 2015.

(51) Int. Cl. A61L 27/36; A61K 35/42; A61L 27/54; A61L 27/52; C12N 5/071

(52) U.S. Cl. CPC A61L 27/3633; A61K 35/42; A61L 27/3687; A61L 27/3691; A61L 27/3666; A61L 27/3679; C12N 2533/92; A61L 27/52; C12N 5/0688; A61L 2400/06; A61L 2430/40; A61L 2430/22; A61L 27/54

(57) ABSTRACT

The presently disclosed subject matter provides for decellularized extracellular matrix (dECM) compositions for reconstructing mucosal tissue of trachea and methods of using the same. In certain embodiments, the composition can be a pre-gel containing dECM derived from tracheal mucosal tissue; a hydrogel obtained by gelling the pre-gel; or a vitrified membrane obtained by drying the hydrogel.

### CROSS-REFERENCE TO RELATED APPLICATIONS

This application is a continuation application of International Application No. PCT/US2016/041566, filed Jul. 8, 2016, which claims priority to U.S. Provisional Application Ser. No. 62/190,130, filed on Jul. 8, 2015, both of which are incorporated by reference herein in their entireties for all purposes.

### TECHNICAL FIELD

The present disclosure relates to compositions comprising decellularized extracellular matrix (dECM), e.g., derived from a tissue, and to methods for reconstructing a tissue using these dECM compositions. For example, and not by way of limitation, the present disclosure provides compositions that include dECM derived from tracheal mucosal tissue and to methods for reconstructing mucosal tissue of trachea using such dECM compositions. In certain embodiments, the present disclosure relates to methods for forming dECM compositions that include a dECM pre-gel derived from mucosal tissue of trachea; a dECM hydrogel, which can be obtained by gelling the pre-gel above; a dECM vitrified membrane, which can be obtained by drying the dECM hydrogel above; and a dECM construct, which can be obtained by removing water from the dECM hydrogel above using various techniques (e.g., vacuum aspiration, plastic compression, etc.). Furthermore, the present disclosure provides methods for tracheal mucosal regeneration using the disclosed dECM compositions, e.g., dECM pre-gel, dECM hydrogel, dECM vitrified membrane, and dECM compressed construct.

### BACKGROUND

The trachea is a conduit that connects the larynx to the lungs, which allows for the passage of air during breathing. The mucosal epithelium covering the luminal surface of trachea has elasticity in the horizontal and vertical directions because the tracheal mucosa is primarily composed of collagen and elastin. The tracheal airway is lined with pseudostratified epithelial cells. Within this pseudostratified tracheal epithelium, there are three main cell types: ciliated cells, goblet cells, and basal cells. The tracheal epithelium serves to warm and humidify air as it enters the respiratory system. It also serves as a filter to protect distal lung structures from damage due to outside environment exposure by mucociliary clearance function of mucus secreting goblet cells and beating ciliated cells.

Self-skin grafting is the most widely used treatment for the critical damage of mucosal epithelium, but it can cause many problems such as decreased mucus flow rate, contraction of the graft, graft detachment, bad smell, etc.

Tissue engineering is the use of a combination of cells, scaffolds, and suitable biochemical factors to repair or replace portions of or whole tissues or organs critically damaged. Engineered scaffolds can be made of polymeric biomaterials such as collagen, hyaluronan, alginate, fibrin, etc., which can provide structural support for cellular attachment and subsequent tissue and/or organ development. To achieve successful tissue and/or organ reconstruction, scaffolds must be similar to the original tissues or organs' microenvironment in their chemical and physical characteristics so scaffolds can interact with the cellular components of the engineered tissues actively to facilitate and regulate their activities by acting like biological or physical cues. However, biomaterials that have been widely used in tissue engineering are limited in their ability to induce or enhance the differentiation and function of specific cells isolated from diverse tissues or organs.

Research on tissue engineering has already been extensively conducted using scaffolds comprising dECM derived from diverse tissues or organs. Scaffolds comprised of specific dECM components derived from a tissue or organ have been shown to have various physical and biological properties depending on the nature of each tissue or organ to isolate it. Thus, dECM scaffolds have been shown to have a powerful effect on inducing or promoting the differentiation and function of tissue-specific cells. Moreover, use of dECM scaffolds can be beneficial as they do not to induce immune graft response that would attack the scaffold. Zang et al. decellularized a whole rat trachea and applied it to tracheal engineering (Zang M. et al., Plast Reconstr Surg. 2012 September: 130(3):532-540). Kutten et al. demonstrated that the decellularized tracheal extracellular matrix supported migration, differentiation, and function of epithelial cells (Kutten, Tissue Eng Part A. 2015 Jan.: 21(12): 75-84). In addition, Baiguera et al. reported improved methods for rat trachea decellularization and crosslinking to increase the mechanical strength of the tracheal scaffold after decellularization process (Baiguera et al., Biomaterials 2014, 35(24):6344-50). However, in previous studies, the whole trachea was decellularized with the goal of whole tracheal reconstruction. At this time, the cartilage surrounding the trachea is very dense, thus the process to remove all the cells from the cartilage is very complex and requires extensive processing using many reagents. These processes cause damage to the cartilage as well as to the surrounding soft tissues, and thus the biological efficacy of the decellularized matrix on tissue or organ reconstruction is not optimal.

Therefore, there remains a need in the art for tissue engineering methods that result in the regeneration of the functional tissue, e.g., mucosal epithelium, more effectively, and for the production of more physiologically relevant in vitro cell culture models.

### SUMMARY

The present disclosure provides for compositions for tissue repair and regeneration, e.g., tracheal mucosal tissue regeneration, and methods of generating and using the same. The present disclosure further provides compositions for generating in vitro cell culture models.

In certain embodiments, the composition for tissue regeneration can be a pre-gel containing dECM. In certain embodiments, the viscosity of the dECM pre-gel can be in a range of about 200 to about 400 Pa·S at a shear rate of 1 s⁻¹ when it is measured at about 15° C. In certain embodiments, the dECM is derived from tracheal mucosal tissue. For example, and not by way of limitation, the dECM present within the composition can be derived from mucosal tissue of porcine trachea. In certain embodiments, the dECM present within the composition can be derived from mucosal tissue of human trachea.

In certain embodiments, the composition for tissue regeneration can be a hydrogel obtained by gelling a pre-gel described herein. In certain embodiments, the dECM hydrogel has a membrane form having a thickness from about 200 to about 2000 μm.

In certain embodiments, the composition for tissue regeneration can be a vitrified membrane obtained by drying a hydrogel described herein. In certain embodiments, the dECM vitrified membrane has a film form having a thickness of about 30 to about 100 μm.

In certain embodiments, the composition for tissue regeneration can be a dECM construct obtained by removing water from a hydrogel described herein. In certain embodiments, water removal is accomplished by applying compression to a hydrogel. In certain embodiments, water removal is accomplished by applying vacuum aspiration to a hydrogel. In certain embodiments, the dECM construct has a thickness of about 30 to about 5000 μm.

The present disclosure further provides methods of tracheal mucosal tissue regeneration. In certain embodiments, the method can include administration or application of a dECM composition disclosed herein, e.g., a dECM pre-gel composition, a hydrogel dECM composition or a vitrified membrane dECM composition, to a patient in need of tracheal mucosal regeneration. In certain embodiments, the composition, e.g., a dECM pre-gel composition, is injected into a tracheal mucosal tissue injury of the patient. In certain embodiments, the composition, e.g., a dECM hydrogel or vitrified membrane composition, is grafted onto a tracheal mucosal tissue injury of the patient.

The present disclosure further provides methods for generating dECM and compositions thereof. In certain embodiments, the dECM present within a composition of the present disclosure can be obtained by treating tissue, e.g., mucosal tissue from a porcine trachea, with at least one detergent such as, but not limited to, sodium dodecyl sulfate and/or polyethylene glycol p-(1,1,3,3-tetra methyl butyl)-phenyl ether. In certain embodiments, the method can further include lyophilizing and/or pulverizing the dECM.

In certain embodiments, a method for generating a dECM pre-gel composition can include treating tissue, e.g., mucosal tissue, with one or more detergents, lyophilizing and/or pulverizing the detergent-treated mucosal tissue to generate dECM, treating the dECM with a proteolytic enzyme in an acidic solution, and neutralizing the acidic solution to obtain the dECM pre-gel composition.

In certain embodiments, a method for generating a dECM hydrogel composition can include treating tissue, e.g., mucosal tissue, with one or more detergents, lyophilizing and/or pulverizing the detergent-treated tissue to generate dECM, treating the dECM with a proteolytic enzyme in an acidic solution, neutralizing the acidic solution to obtain the dECM pre-gel composition, and gelling the dECM pre-gel composition to obtain the dECM hydrogel composition.

In certain embodiments, a method for generating a dECM vitrified membrane composition can include treating tissue, e.g., mucosal tissue, with one or more detergents, lyophilizing and/or pulverizing the detergent-treated tissue to generate dECM, treating the dECM with a proteolytic enzyme in an acidic solution, neutralizing the acidic solution to obtain a dECM pre-gel composition, gelling the dECM pre-gel composition to obtain a dECM hydrogel composition, and drying the dECM hydrogel composition to obtain the dECM vitrified membrane composition.

In certain embodiments, the dECM derived from the tissue, e.g., tracheal mucosal tissue, is added to an acidic solution at a concentration from about 0.3 and about 4 w/v %. In certain embodiments, the acidic solution can include acetic acid, hydrochloric acid solution, or a combination thereof. In certain embodiments, the proteolytic enzyme can be pepsin, a matrix metalloproteinase, or a combination thereof.

The present disclosure further provides dECM compositions for analyzing tissue in vitro that comprise dECM derived from a tissue, where the dECM is in the form of a hydrogel or a vitrified membrane. For example, and not by way of limitation, the present disclosure provides dECM compositions for analyzing tracheal mucosal tissue in vitro that comprise dECM derived from tracheal mucosal tissue, where the dECM is in the form of a hydrogel or a vitrified membrane. In certain embodiments, the dECM hydrogel has a thickness from about 200 to about 2000 μm. In certain embodiments, the dECM vitrified membrane has a thickness of about 30 to about 100 μm.

### BRIEF DESCRIPTION OF THE FIGURES

FIG. 1 is a schematic diagram showing the process of decellularization using porcine tracheal mucosal tissue and the effects of the dECM compositions on cell viability and differentiation.

FIG. 2 is a schematic diagram showing the fabrication process of the pre-gel and hydrogel containing dECM derived from tracheal mucosal tissue.

FIG. 3 shows the rheological behavior of the dECM pre-gel.

FIG. 4 shows the effect of dECM composition on the viability (green: live cells, red: dead cells) and proliferation of the lung fibroblasts.

FIG. 5 is a schematic diagram of the air-liquid interface culture (ALI-culture) for inducing the differentiation of the mucociliary tracheal epithelium from human tracheal epithelial cells using the hydrogel comprising dECM derived from tracheal mucosal tissue.

FIG. 6 shows the assessment of the ciliated cells formation by the hydrogel comprising dECM derived from tracheal mucosal tissue on the ALI-culture.

FIG. 7 shows the assessment of goblet cell formation and mucus secretion by a hydrogel comprising dECM derived from tracheal mucosal tissue on the ALI-culture.

FIG. 8 is a schematic diagram of the experiment for the assessment of the mucus flow by a differentiated tracheal epithelium on a hydrogel comprising dECM derived from tracheal mucosal tissue.

FIG. 9 shows the trajectory lines of the fluorescence microspheres movement on the differentiated tracheal epithelium by the mucus flow.

FIG. 10 shows the quantitative analysis of fluorescence microspheres movement (the speed of the microspheres and meandering index) on the differentiated tracheal epithelium.

FIG. 11 is the schematic diagram of the fabrication process for the vitrified membrane comprising dECM derived from porcine tracheal mucosal tissue.

FIG. 12 shows the viability and proliferation of embryonic fibroblasts encapsulated in dECM hydrogel comprising dECM derived from tracheal mucosal tissue.

FIG. 13 shows a design of a transwell to apply the dECM vitrified membrane to ALI-culture of tracheal epithelial cells.

FIG. 14 shows the effect of dECM composition on the gene and protein expression of tight junction and cilia markers, epithelial cell markers, and transcription factors.

FIGS. 15A-15F show the decellularization of the tracheal mucosal tissue and its biochemical analysis. FIG. 15A shows microscopic images of native and decellularized tracheal mucosal tissue. FIG. 15B shows the DNA contents and ECM components (Collagen and GAGs) of native and decellularized tracheal mucosal tissue. FIG. 15C shows an SEM image of the surface of the freeze-dried 3% (w/v) tmdECM (trachea mucosal dECM) hydrogel. FIGS. 15D, E and F show the rheological properties of Col-1 and tmdECM pre-gels. FIG. 15D shows the viscosity of the pre-gels at 15° C. FIG. 15E shows the gelation kinetics of the pre-gel from 4° C. to 37° C. (at increments of 5° C./min until 37° C. was reached, followed by maintenance at 37° C. for 30 min). FIG. 15D shows the complex modulus of the pre-gel at 37° C.

FIGS. 16A-16C show the effect of tmdECM on tracheal epithelium regeneration in vivo. FIG. 16A shows the process for implantation of ECM-coated scaffolds. FIG. 16B shows the histological analysis of tracheal epithelium regeneration by Hematoxylin and eosin (H&E) staining 2 weeks post implantation. FIG. 16C shows an ex vivo cilia motility assay showing the trajectories of microspheres and the analysis of the speed, velocity and meandering index of the microspheres movement ex vivo.

### DETAILED DESCRIPTION

The present disclosure provides decellularized extracellular matrix (dECM) compositions and methods of use thereof. The present disclosure further provides methods for generating the dECM compositions. In particular, the present disclosure provide compositions containing dECM derived from tissue, e.g., tracheal mucosal tissue, and to methods for reconstructing tissue, e.g., mucosal tissue of trachea, using these dECM compositions.

As shown below, various studies were performed to validate that the disclosed dECM compositions can mimic the microenvironment of the tracheal mucosa and methods have been developed for inducing tracheal mucosa regeneration using the disclosed dECM compositions. As described herein, the dECM pre-gel, hydrogel, and vitrified membrane compositions can be very effective in regenerating functional tracheal mucosa.

Accordingly, the present disclosure provides dECM compositions that include dECM pre-gel, dECM hydrogel, and dECM vitrified membrane compositions for the repair and/or regeneration of tissue, e.g., tracheal mucosa tissue. The present disclosure further provides methods for generating such compositions. In certain embodiments, the present disclosure provides dECM compositions for analyzing tracheal mucosa ex vivo that can include a pre-gel, which contains dECM derived from trachea mucosa; a hydrogel, which can be obtained by gelling the pre-gel above; or a vitrified membrane, which can be obtained by drying the hydrogel above.

The present disclosure further provides methods for the repair and/or regeneration of tissue, e.g., tracheal mucosa tissue. In certain embodiments, methods of the present subject matter can include the administration or application of the compositions disclosed herein to patients in need of tissue repair and/or regeneration, e.g., tracheal mucosal regeneration.

### Compositions

The present disclosure provides compositions generated from dECM. In certain embodiments, the compositions of the present disclosure can include dECM derived from parts of the respiratory system that include, but are not limited to, the trachea, nasal passages, bronchi, bronchioles, and alveoli. For example, and not by way of limitation, the dECM can be derived from the trachea, e.g., from tracheal mucosal tissue. In certain embodiments, the dECM can be derived from other organs that include, but are not limited to, the skin, eye, heart, liver, intestine, stomach, placenta, cervix, brain, and bone.

In certain embodiments, the dECM can be derived from the tissue of any mammal, e.g., from the tracheal mucosal tissue of any mammal. In certain embodiments, the dECM can be obtained by decellularizing tissue, e.g., tracheal mucosal tissue, isolated from mammals including, but not limited to, humans, porcine, cattle, rabbits, dogs, goats, sheep, chickens, horses, etc.

In certain embodiments, the compositions of the present disclosure can be used for tissue repair and/or tissue regeneration, e.g., for tracheal mucosa regeneration and/or repair. In certain embodiments, the compositions for tissue regeneration and/or repair can include a pre-gel containing dECM derived from a tissue. For example, and not way of limitation, compositions for tracheal mucosa regeneration and/or repair can include a pre-gel containing dECM derived from tracheal mucosal tissue. In certain embodiments, the dECM pre-gel composition can have the characteristics of a homogeneous solution with suitable viscoelasticity and flow behavior for injection to the injured area for clinical treatment. For example, and not by way of limitation, the viscosity of the dECM pre-gel composition can be in a range between about 200 to about 400 Pa·S at a shear rate of 1 s⁻¹ when it is measured at 15° C. In certain embodiments, the viscosity of the dECM pre-gel composition can be from about 200 to about 250 Pa·S, from about 200 to about 300 Pa·S, from about 200 to about 350 Pa·S, from about 250 to about 400 Pa·S, from about 300 to about 400 Pa·S or from about 350 to about 400 Pa·S.

In certain embodiments, the dECM pre-gel can contain components that are present in tissue from which was it derived. In certain embodiments, the dECM pre-gel can contain components that are present in tracheal mucosal tissue, e.g., to mimic the characteristics of the tracheal mucosal tissue and its complex organization and function. For example, and not by way of limitation, the dECM pre-gel can include collagen, glycosaminoglycan, laminin, elastin, non-collagenous protein and the like.

In certain embodiments, the compositions for tissue regeneration and/or repair. e.g., compositions for tracheal mucosa regeneration and/or repair, can include a hydrogel that includes dECM. For example, and not by way of limitation, the hydrogel can be obtained by gelling a dECM pre-gel composition disclosed herein. In certain embodiments, the dECM hydrogel can be obtained by gelling the pre-gel at 37° C. The gelled structure can have a resulting thickness of about 200 to about 2000 μm. For example, and not by way of limitation, the dECM hydrogel composition can have a thickness from about 300 to about 2000 μm, from about 400 to about 2000 μm, from about 500 to about 2000 μm, from about 600 to about 2000 μm, from about 700 to about 2000 μm, from about 800 to about 2000 μm, from about 900 to about 2000 μm, from about 1000 to about 2000 μm, from about 1200 to about 2000 μm, from about 1400 to about 2000 μm, from about 1600 to about 2000 μm, from about 1800 to about 2000 μm, from about 200 to about 1800 μm, from about 200 to about 1600 μm, from about 200 to about 1400 μm, from about 200 to about 1200 μm, from about 200 to about 1000 μm, from about 200 to about 900 μm, from about 200 to about 800 μm, from about 200 to about 700 μm, from about 200 to about 600 μm, from about 200 to about 500 μm, from about 200 to about 400 μm or from about 200 to about 300 μm.

In certain embodiments, the compositions for tissue regeneration and/or repair. e.g., compositions for tracheal mucosa regeneration and/or repair, can include a vitrified membrane obtained by drying the dECM hydrogel disclosed herein. In certain embodiments, the dECM vitrified membrane composition can be obtained by drying the dECM hydrogel in the form of a membrane, and can be in a film form having a thickness of about 30 to about 100 μm. For example, and not by way of limitation, the vitrified membrane can have a thickness from about 40 to about 100 μm, from about 50 to about 100 μm, from about 60 to about 100 μm, from about 70 to about 100 μm, from about 80 to about 100 μm, from about 90 to about 100 μm, from about 30 to about 90 μm, from about 30 to about 80 μm, from about 30 to about 70 μm, from about 30 to about 60 μm, from about 30 to about 50 μm or from about 30 to about 40 μm.

As disclosed herein, the dECM pre-gel, hydrogel, or vitrified membrane compositions derived from tracheal mucosal tissue can function as a scaffold for tracheal mucosa regeneration by mimicking the microenvironment of the tracheal mucosa. For example, and not by way of limitation, these dECM compositions derived from tracheal mucosal tissue, including the pre-gel, hydrogel, or vitrified membrane compositions, can mimic the characteristics of tracheal mucosal tissue with its complex organization and microenvironment. Therefore, in certain embodiments, the disclosed compositions can be used as a valuable scaffold for the regeneration and/or repair of the functional tracheal mucosal epithelium.

In certain embodiments, the hydrogel or vitrified membrane described above can be used in the analysis of tracheal mucosal tissue ex vivo. In certain embodiments, the compositions described herein can be used to generate in vitro cell culture models, which can provide instructive microenvironmental cues that allow cultured cells to express more physiological phenotypes.

### Methods for Generating the Compositions

The present disclosure provides methods for generating the disclosed dECM compositions. In certain embodiments, the present disclosure provided methods for obtaining compositions generated from dECM obtained from a tissue, disclosed herein. In certain embodiments, the present disclosure provided methods for obtaining compositions generated from dECM obtained from the mucosal tissue of trachea. For example, and not by way of limitation, the disclosed dECM can be obtained from the mucosal tissue of a porcine trachea.

In certain embodiments, the methods of the present disclosure include decellularizing tissue, e.g., tracheal mucosal tissue. In certain embodiments, a cellular disruption medium can be used to decellularize the tissue, e.g., tracheal mucosal tissue. In certain embodiments, the cellular disruption medium can include at least one detergent. Selection of detergent type and concentration can be based partly on its preservation of the structure, composition, and biological activity of the extracellular matrix. For example, but not by way of limitation, the detergent can be an anionic or a non-ionic detergent. Non-limiting examples of such detergents include sodium dodecyl sulfate (SDS) and Triton X-100. For example, SDS and Triton-X can be used at a concentration of 1% in PBS. In certain embodiments, the tissue is treated with a combination of different classes of detergents, for example, a nonionic detergent, Triton X-100, and an anionic detergent, sodium dodecyl sulfate, to disrupt cell membranes and aid in the removal of cellular debris from tissue. In certain embodiments, the tissue is initially treated with SDS, followed by treatment with Triton X-100. In certain embodiments, the cellular disruption medium can include one or more detergents at a concentration of about 0.1% to about 10%, e.g., from about 0.5% to about 10%, from about 1% to about 10%, from about 2% to about 10%, from about 3% to about 10%, from about 4% to about 10%, from about 5% to about 10%, from about 6% to about 10%, from about 7% to about 10%, from about 8% to about 10%, from about 0.5% to about 10%, from about 9% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1% or from about 0.1% to about 0.5%. In certain embodiments, the detergent in present in the disruption medium at a concentration of about 1%. In certain embodiments, the dECM described above can be obtained by performing an additional process of freeze-drying (i.e., lyophilizing) and/or pulverizing after treatment with the cellular disruption medium described above.

In certain embodiments, the pre-gel described above can be obtained by (a) treatment of a dECM derived from a tissue, e.g., tracheal mucosa, with a proteolytic enzyme in an acidic solution; and (b) titration of the acidic solution obtained to a neutral solution by the addition of a base. In certain embodiments, the neutral pH has a pH of about 6 to about 8, e.g., about 7.

In certain embodiments, in step (a), the dECM derived from a tissue, e.g., tracheal mucosal tissue, described above can be used at a proper amount in the suitable range for tracheal mucosal regeneration. In certain embodiments, the dECM, e.g., derived from tracheal mucosal tissue described above, can be dissolved in an acidic solution at a concentration between about 0.3 and about 4% weight by volume (w/v). For example, and not by way of limitation, the dECM can be dissolved into an acidic solution at a concentration from about 0.5% to about 4% w/v, from about 1% to about 4% w/v, from about 1.5% to about 4% w/v, from about 2% to about 4% w/v, from about 2.5% to about 4% w/v, from about 3% to about 4% w/v, from about 3.5% to about 4% w/v, from about 0.3 and about 3.5% w/v, from about 0.3 and about 3.5% w/v, from about 0.3 and about 3% w/v, from about 0.3 and about 2.5% w/v, from about 0.3 and about 2% w/v, from about 0.3 and about 1.5% w/v, from about 0.3 and about 1% w/v or from about 0.3 and about 0.5% w/v.

In certain embodiments, the proteolytic enzymes used to obtain the pre-gel can include enzymes that perform a digestive function. In certain embodiments, the proteolytic enzymes can be pepsin, a matrix metalloproteinase or the like. Non-limiting examples of matrix metalloproteinases are disclosed in Visse and Nagase Circulation Research (2003) 92: 827-839. For example, 1 mg/mL pepsin in 3% acetic acid can be used. The amount of the proteolytic enzyme can differ depending on the contents of the dECM, for example, and not by way of limitation, the range of about 1 to about 5 mg of proteolytic enzyme can be used for about 100 mg of dECM. In certain embodiments, the proteolytic enzyme can be used at a ratio of about 1:100 proteolytic enzyme to dECM, at a ratio of about 1:50 proteolytic enzyme to dECM, at a ratio of about 3:100 proteolytic enzyme to dECM, at a ratio of about 1:25 proteolytic enzyme to dECM or at a ratio of about 1:20 proteolytic enzyme to dECM.

In certain embodiments, the acidic solution provides the acidic condition for dissolving the dECM and for facilitating the action of the proteolytic enzymes. In certain embodiments, an acetic acid or hydrochloric acid solution can be used as the acidic solution described above. In certain embodiments, the acidic solution can have a pH in the range of about 2 to about 4, e.g., from about 2.5 to about 4, from about 3 to about 4, from about 3.5 to about 4, from about 2 to about 4, from about 2 to about 3.5, from about 2 to about 3 or from about 2 to about 2.5. In certain embodiments, the acidic solution can have a pH from about 2.5 to about 3.

In certain embodiments, step (b) can be carried out by neutralizing the acidic solution by adding a base. In certain embodiments, the base used to neutralize the acid can be sodium hydroxide (NaOH). For example, and not by way of limitation, sodium hydroxide can be used in a sufficient amount to adjust the acid to about pH 7. In certain embodiments, the concentration of the base solution used to neutralize the acidic solution can be between 9M and 11M, e.g., 10M.

In certain embodiments, the acidic solution, e.g., a hydrochloric acid or acetic acid solution, can have a concentration of about 0.01 to about 10 M, e.g., from about 0.5 to about 10 M, from about 1.5 to about 10 M, from about 2 to about 10 M, from about 2.5 to about 10 M, from about 3 to about 10 M, from about 3.5 to about 10 M, from about 4 to about 10 M, from about 4.5 to about 10 M, from about 5 to about 10 M, from about 5.5 to about 10 M, from about 6 to about 10 M, from about 6.5 to about 10 M, from about 7 to about 10 M, from about 7.5 to about 10 M, from about 8 to about 10 M, from about 8.5 to about 10 M, from about 9 to about 10 M, from about 9.5 to about 10 M, from about 0.1 to about 9.5 M, from about 0.1 to about 9 M, from about 0.1 to about 8.5 M, from about 0.1 to about 8 M, from about 0.1 to about 7.5 M, from about 0.1 to about 7 M, from about 0.1 to about 6.5 M, from about 0.1 to about 6 M, from about 0.1 to about 5.5 M, from about 0.1 to about 5 M, from about 0.1 to about 4.5 M, from about 0.1 to about 4 M, from about 0.1 to about 3.5 M, from about 0.1 to about 3 M, from about 0.1 to about 2.5 M, from about 0.1 to about 2 M, from about 0.1 to about 1.5 M, from about 0.1 to about 1 M or from about 0.1 to about 0.5 M.

In certain embodiments, the pre-gel form of the dECM composition can have the characteristics of a homogeneous solution with viscoelasticity that has a suitable flow behavior for injection to the injured area in the clinical treatment. The viscosity can be adjusted by appropriately controlling the amount of aqueous medium. Non-limiting examples of aqueous medium include distilled water, purified water, water for injection, PBS, physiological saline, etc. In certain embodiments, the viscosity of the dECM pre-gel composition can be in a range of about 200 to about 400 Pa·S at a shear rate of 1 s⁻¹ when it is measured at 15° C.

In certain embodiments, a dECM hydrogel composition can be obtained by gelling a dECM pre-gel composition, disclosed herein, at a temperature of about 20 to about 40° C. In certain embodiments, the temperature can be from about 30 to about 40° C. or from about 35 to about 40° C., e.g., gelling at 37° C. In certain embodiments, the gelling of the dECM pre-gel composition to obtain the vitrified membrane can be performed at 37° C. for about 30 minute to 2 hours. In certain embodiments, the dECM hydrogel composition has a thickness of about 200 to about 2000 μm.

In certain embodiments, the a dECM vitrified membrane composition described above can be obtained by drying the hydrogel. The drying described above can be carried out between 20-25° C. In certain embodiments, the drying of the hydrogel to obtain the vitrified membrane can be performed at room temperature (RT) for about 12 to about 72 hours. In certain embodiments, the drying time is about 24 hours, but the drying time is not limited to this condition. In certain embodiments, once dried, the dECM vitrified membrane composition is a thin film with a thickness of about 30 to about 100 μm. The thickness of the dECM vitrified membrane is dependent upon the initial volume of gel to be dried.

### Methods of Use

The present disclosure further provides methods for tissue regeneration and/or repair that can include administration or application of the compositions described herein to patients with tissue defects. In certain embodiments, the present disclosure provides methods for tracheal tissue regeneration that can include administration or application of a dECM composition derived from tracheal mucosal tissue to patients with tracheal mucosal defects.

In certain embodiments, a method for regeneration and/repair of a tissue, e.g., tracheal mucosal tissue, can include applying a dECM pre-gel composition to a tissue injury, e.g., a mucosal injury of the trachea. For example, and not by way of limitation, the dECM vitrified membrane composition can be injected into the mucosal injury. In certain embodiments, the methods can further include the gelling of the dECM vitrified membrane composition at body temperature to function as a scaffold for regeneration of tracheal mucosal tissue.

In certain embodiments, a method for regeneration and/repair of a tissue, e.g., tracheal mucosal tissue, can be carried out using a dECM hydrogel composition or dECM vitrified membrane composition. For example, and not by way of limitation, the hydrogel or vitrified membrane described herein can be applied as a patch or graft overlying the tissue injury, e.g., mucosal injury of trachea. In certain embodiments, the dECM hydrogel composition can be obtained by gelling the pre-gel at a temperature of about 20 to about 40° C., e.g., at 37° C. In certain embodiments, the hydrogel can be a membrane form having a thickness of about 200 to about 2000 μm. In certain embodiments, the dECM vitrified membrane composition can be obtained by drying the hydrogel, it can be a film form having a thickness of about 30 to about 100 μm. The drying described above can be carried out at RT (e.g., about 20 to about 25° C.) for about 12 to about 48 hours, e.g., 12 hours.

In certain embodiments, a dECM composition derived from tissue (e.g., tracheal mucosal tissue), e.g., a dECM pre-gel composition, a dECM hydrogel composition, or a dECM vitrified membrane composition can be administered at a suitable range for tissue regeneration. In certain embodiments, the dECM composition can be administered in a range of about 1 to about 15 mg/cm² of injured tissue. In certain embodiments, the dose is in the range of about 1 to about 10 mg, about 2 to about 9, about 3 to about 8 or about 4 to about 7, but can vary depending on the patient's condition and extent of the damage.

In certain embodiments, a dECM-derived composition, e.g., a dECM hydrogel composition, disclosed herein can also be used as a scaffold material for three-dimensional (3D) bio-printing. For example, and not by way of limitation, cells can be encapsulated in a dECM composition of the present disclosure and the mixture can be 3D printed to generate complex three dimensional structures for regenerative medicine and assay development.

In certain embodiments, a composition of the present disclosure can be implanted on a scaffold, and used in methods for tissue regeneration, e.g., tracheal mucosal tissue generation. In certain embodiments, a composition of the present disclosure, e.g., a hydrogel, can be mounted onto a scaffold, e.g., a mesh. Non-limiting examples of such scaffolds can include polymers, which can include poly(hydroxyl acids), poly(lactic) acids, polyanhydrides, polyorthoesters, polyamides, polyalkylenes, polyalkylene glycols, polyalkylene oxides, polyvinyl alcohols, polyvinyl ethers, polyvinyl esters, polyvinyl alcohols, poly(butyric acid), polyvinylpyrrolidone, poly(valeric acid), polycaprolactone, poly(lactide-co-caprolactone), poly(dimethyl)siloxane and poly(acrylonitrile). For example, and not by way of limitation, the scaffold can include polycaprolactone (PCL). In certain embodiments, the scaffold can be generated by a 3D printer. In certain embodiments, the scaffold can be generated by replica molding techniques.

In certain embodiments, the vitrified membrane can be used in the clinic or for analysis ex vivo (e.g., assay chip) because the membrane has a scaffold structure that cells can easily be affixed to and because the membrane has the form of a film with high density of fiber and good mechanical strength. In certain embodiments, the composition in the form of vitrified membrane is highly elastic with good mechanical strength. Even when re-hydrated in cell culture media, the vitrified membrane can maintain these properties of strength and elasticity and is sufficiently durable for handling with sharp forceps. These characteristics of the vitrified membrane facilitate its use in clinical applications, as clinicians can easily manipulate the membrane using surgical equipment such as forceps.

The present disclosure also provides methods for the use of hydrogel or the vitrified membrane described above for the analysis of tracheal mucosal tissue ex vivo. In certain embodiments, a pre-gel derived from tracheal mucosal tissue; a hydrogel obtained by gelling the pre-gel described above; or a vitrified membrane obtained by drying the hydrogel described above can be used for the analysis of tracheal mucosal tissue ex vivo.

In certain embodiments, the present disclosure provides methods for using the compositions disclosed herein, e.g., the hydrogel, vitrified membrane, and dECM pre-gel, for generating in vitro cell culture models. For example, and not by way of limitation, cells can be cultured within the disclosed compositions to replicate physiological conditions and/or to induce the cultured cells to express their native morphological and functional phenotypes. In certain embodiments, compositions of the present disclosure derived from tracheal mucosal tissue can be used to culture tracheal cells in vitro.

The present disclosure could also be used to develop cellular assays and tissue engineering scaffolds not only for the trachea but also for other parts of the respiratory system including nasal passages, bronchi, bronchioles, and alveoli. For example, and not by way of limitation, the compositions of the present disclosure can include dECM derived from other parts of the respiratory system such as the nasal passages, bronchi, bronchioles, and alveoli.

The present disclosure is applicable to creating more physiological cell culture platforms, biological assays, and tissue engineering scaffolds for other organs. Non-limiting examples of such organs include the skin, eye, heart, liver, intestine, stomach, placenta, cervix, brain, and bone.

The present disclosure of dECM-derived material can also be used to generate a vehicle for carrying cells for cell therapy applications. For example, and not by way of limitation, the dECM compositions can further include one or more cell type, including epithelial cells, endothelial cells, muscle cells, neurons, fibroblasts, stem cells, immune cells, and more. The disclosed compositions can enable cells to retain their native functionalities and/or provide new opportunities to engineer cellular properties and functions for increased therapeutic efficacy and better clinical outcomes.

### EXAMPLES

The present disclosure will be explained in more detail in the examples below. However, the following examples are to illustrate the present disclosure and the scope of the present disclosure is not to be limited by the following examples.

### Example 1: The Fabrication and Evaluation of the Pre-Gel and Hydrogel Comprising the dECM Derived from the Mucosal Tissue of Trachea

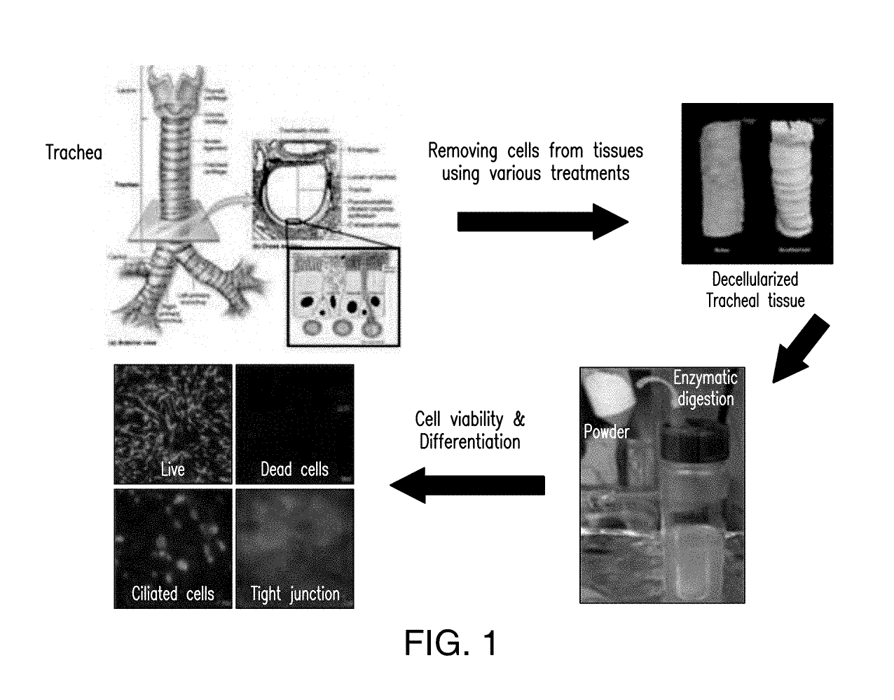

The pre-gel and hydrogel comprising the dECM derived from mucosal tissue of porcine trachea were fabricated, and their effects on the cell viability, proliferation, differentiation, and function were evaluated. These processes are represented as a schematic diagram in FIG. 1.

(1) The Fabrication of the Pre-Gel and Hydrogel Comprising the dECM

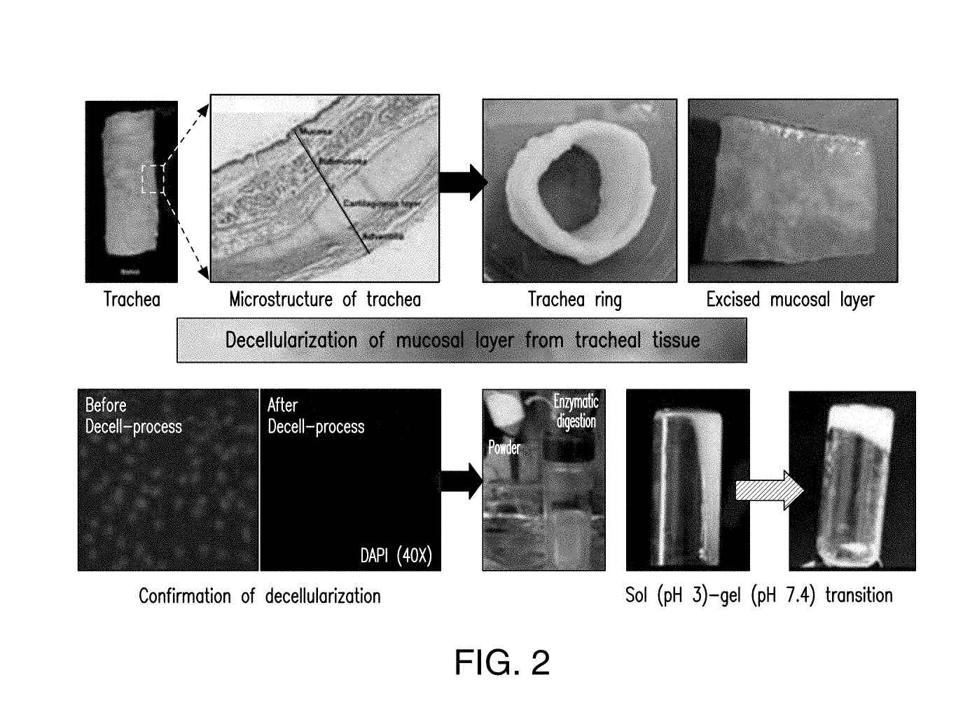

The mucosal tissues of the porcine trachea (about 6 months of age) were isolated and washed with distilled water. After slicing the mucosal tissues into about 1 mm pieces, themucosal pieces (700 mg) were stirred in 1% sodium dodecyl sulfate in phosphate buffer (PBS) (500 ml) for 48 hours, and then, treated with 1% Triton X-100 (500 ml). The mucosal pieces were then thoroughly washed with PBS and the residual amount of DNA was assessed with a nuclear fluorescent stain, DAPI. The decellularized mucosa pieces were lyophilized, and then pulverized. Subsequently, the composition in the form of the pre-gel and hydrogel was fabricated using the decellularized mucosal tissue (FIG. 2). That is, the dECM powder (300 mg) was added to 0.5M acetic acid solution (10 ml) including pepsin (1 mg/ml), and the solution was stirred at RT for 72 hours. The resulting solubilized dECM solution was acidic in nature and was adjusted to physiological pH (about pH 7) using 10M NaOH solution while maintaining the temperature below 10° C. The pH-adjusted dECM solution (the dECM pre-gel) was stored at 4° C. To form the dECM hydrogel, the pre-gel was incubated at 37° C.

(2) Characterization of the Rheological Behavior of the dECM Pre-Gel

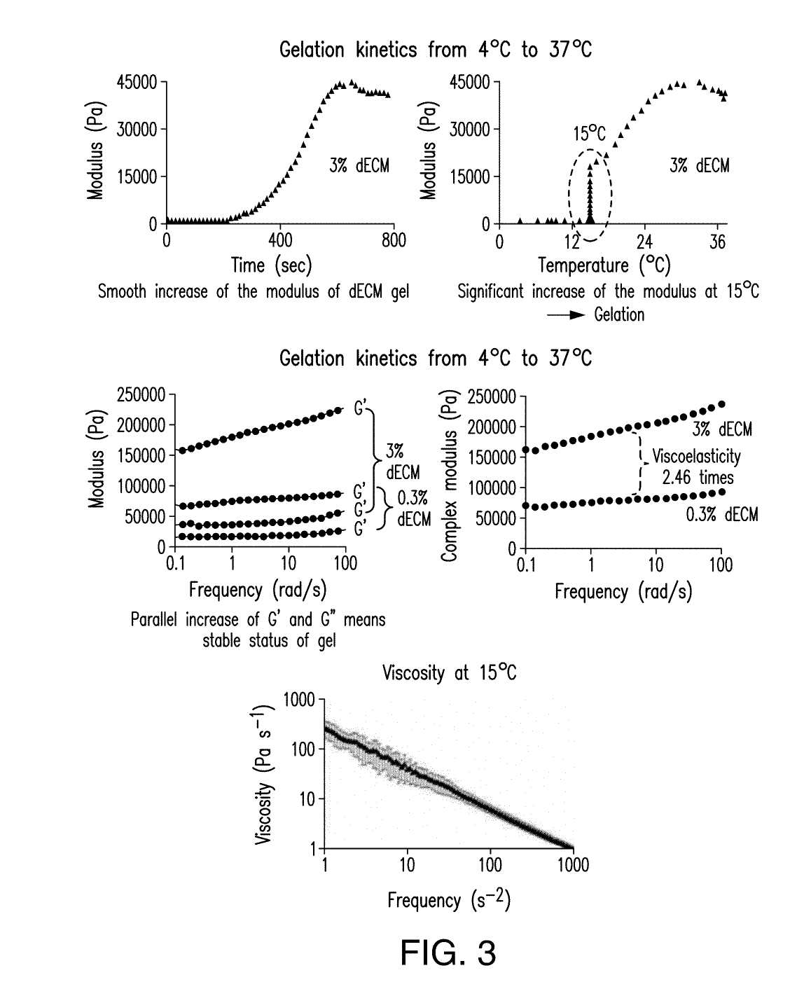

Rheological investigations of the fabricated dECM pre-gel from (1) were conducted using a rheometer. The modulus of elasticity was measured at each temperature while the dECM pre-gel was subjected to a temperature ramp in the range of 4−37° C. with a increment rate of 5° C./min, and it was confirmed that the dECM pre-gel stably undergoes gelation (FIG. 3; gelation kinetics).

The shear storage (G′) and shear loss (G″) moduli of the dECM pre-gels were measured, it was confirmed that the dECM pre-gel is very stable against an external stimulus, and has a high viscoelasticity (FIG. 3. Dynamic modulus). The viscosity of the 3% (w/v) dECM pre-gel was measured at 15° C., and the value at 1 s⁻¹ shear was represented in a range of 200-400 Pa. S, which has a proper flow behavior to be injected to the mucosal injury of trachea (FIG. 3; Viscosity).

(3) Evaluation of the dECM Pre-Gel and Hydrogel

(3-1) Cell Viability and Proliferation Test

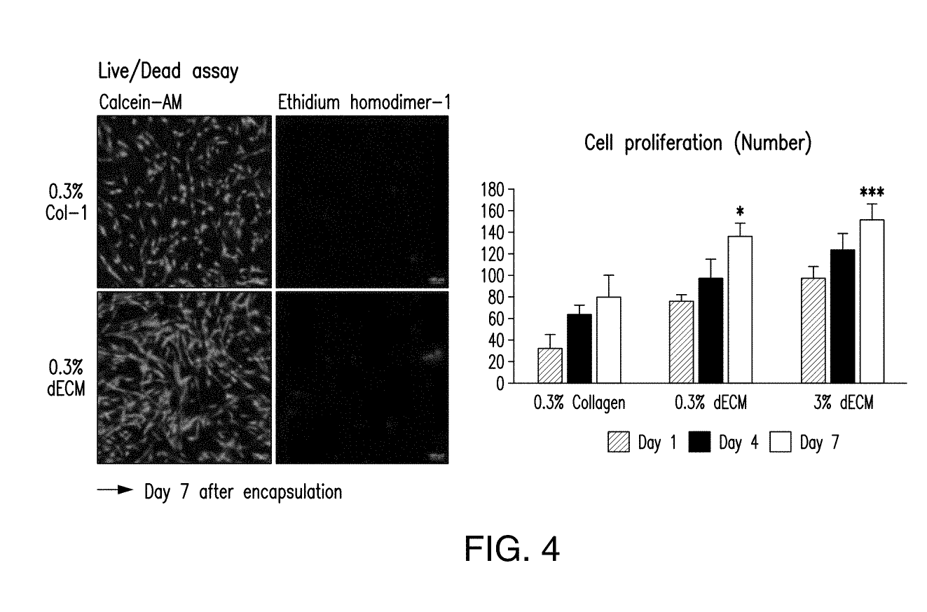

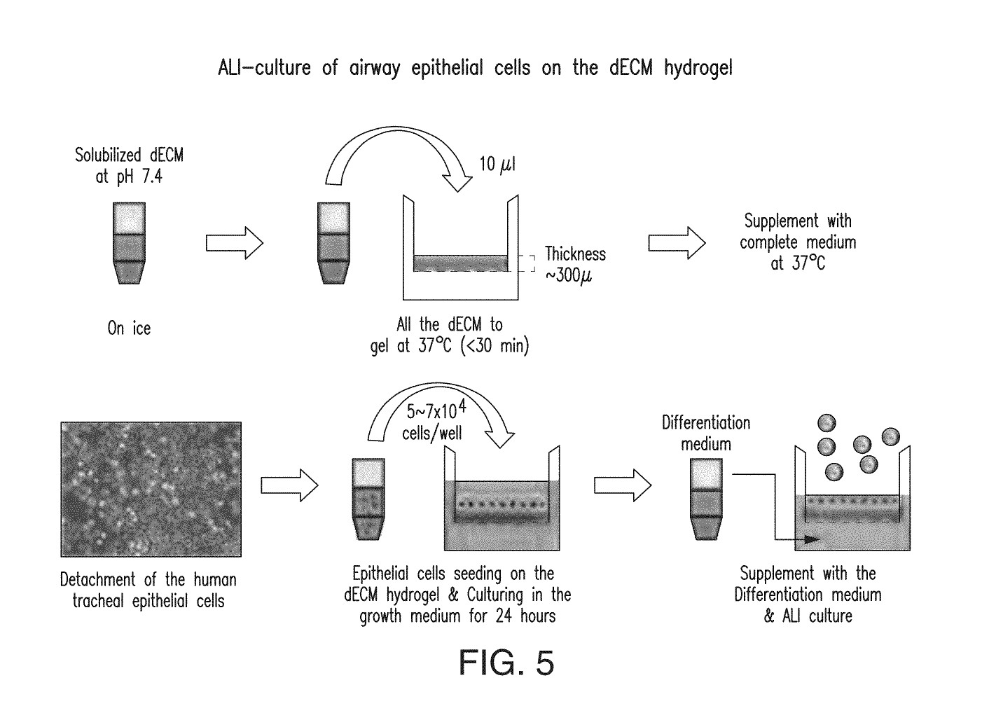

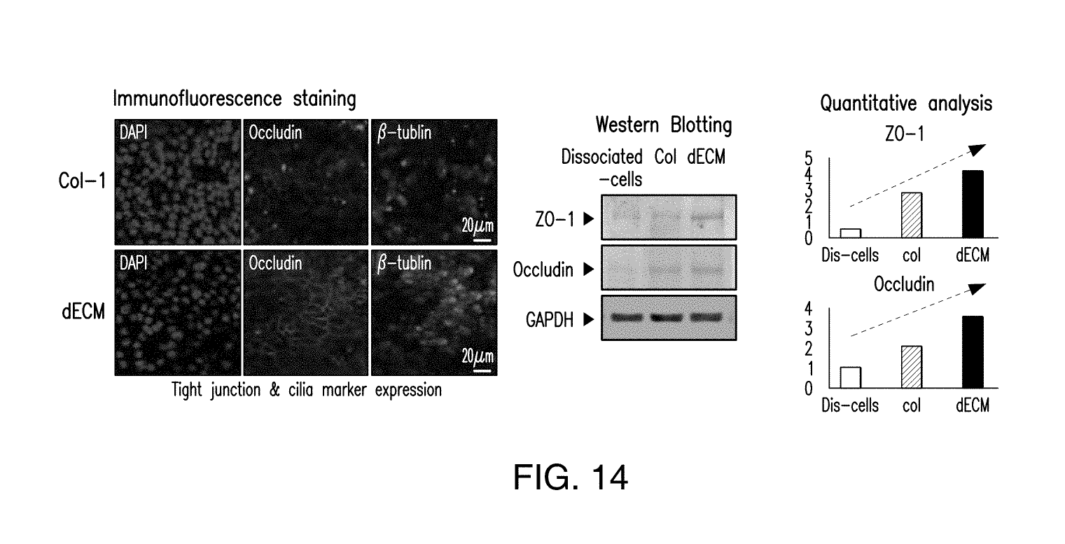

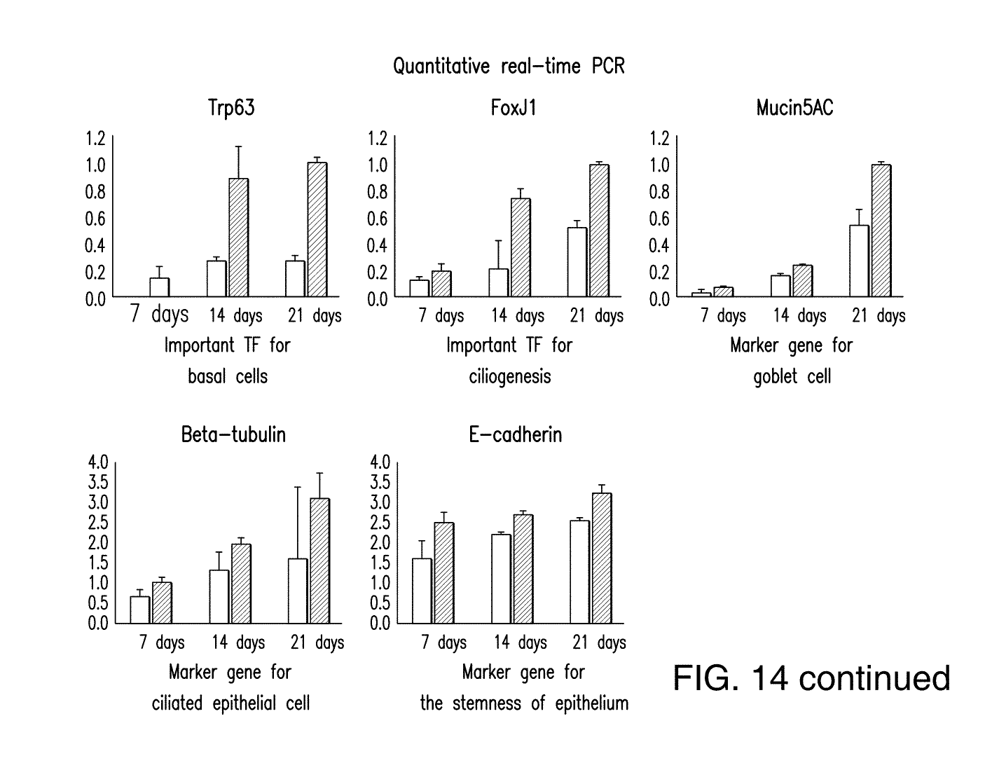

To evaluate the effect of the dECM on cell viability, LIVE/DEAD Cell Viability Assays was conducted. Lung fibroblasts (HFL1, ATCC CCL-153) were mixed with pH-adjusted dECM pre-gel at the concentration of 2×10⁶ cells/ml on ice. The prepared cell-dECM pre-gel mixture was injected onto the 24 well-plate, and crosslinked by incubation at 37° C. Dulbecco's modification of Eagle medium (DMEM)/10% fetal bovine serum (FBS) was supplemented into each well, and the cells were cultured at 37° C., in 5% CO₂ for 1, 4, and 7 days to analyze cell viability using a Live/Dead assay. Collagen was used as a control for comparative analysis, because it is the most abundant component of ECM in our body. Cell viability and proliferation were significantly higher in the dECM than that of collagen (FIG. 4). The protein and gene expressions of tight junction and cilia markers, epithelial cell markers, and important transcription factors were also higher in the dECM than that of collagen (FIG. 14).

(3-2) Differentiation of Ciliated Cells and Goblet Cells

Human tracheal epithelial cells (hTEpCs; PromoCell; Heidelberg, Germany) were used to examine the effect of the dECM hydrogel on differentiation of the ciliated cell, which is one of the major epithelial cell types in human adult lung.

The dECM pre-gel from (1) was loaded onto the insert of transwell, and incubated at 37° C. for fabricating the hydrogel. hTEpCs were seeded onto the hydrogel in the insert, and cultured for 1d. Then, the media in the insert was removed for exposure of the cells to air for air-liquid interface culture (ALI-culture), and differentiation medium (B-ALI differentiation medium, Lonza, Walkersville, Md.) was added into the bottom well of the transwell for inducing the differentiation of tracheal epithelium. Collagen was also used as a control for comparative analysis.

Differentiation of ciliated cells was observed on the dECM hydrogel at 14 d after ALI-culture, while the ciliated cells were not observed at this time point on the collagen hydrogel. Formation of ciliated cells was observed on the collagen hydrogel at 17 d after ALI-culture. However, the number of the beating ciliated cells were significantly higher on the dECM hydrogel than that of the collagen hydrogel over time (FIG. 6).

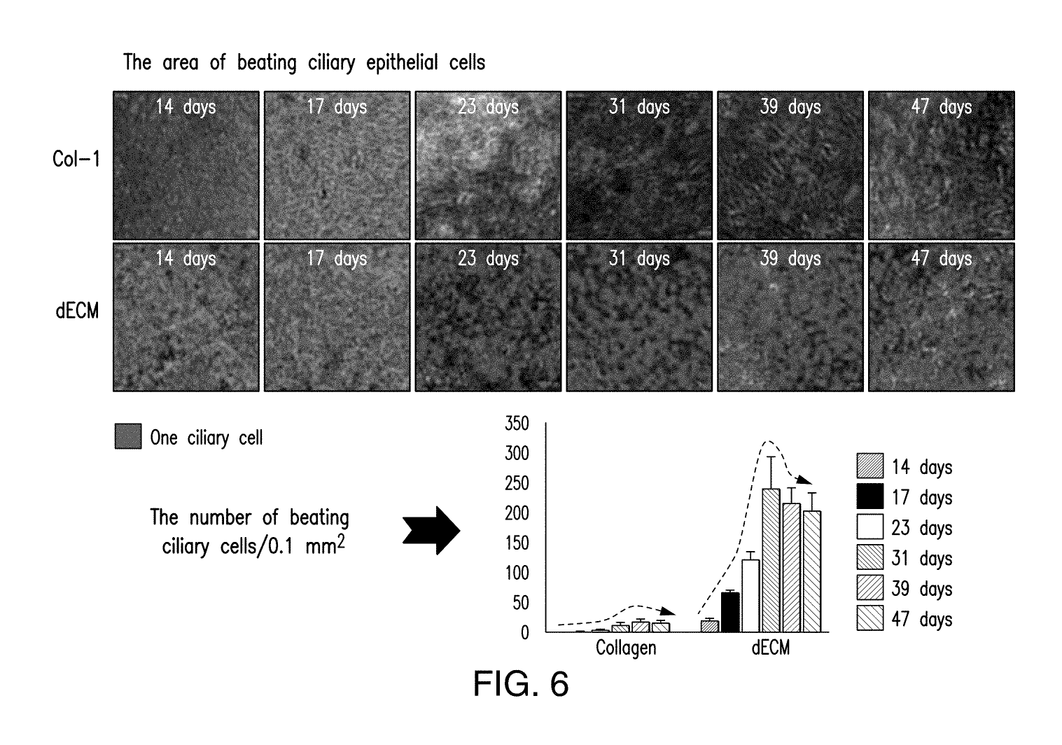

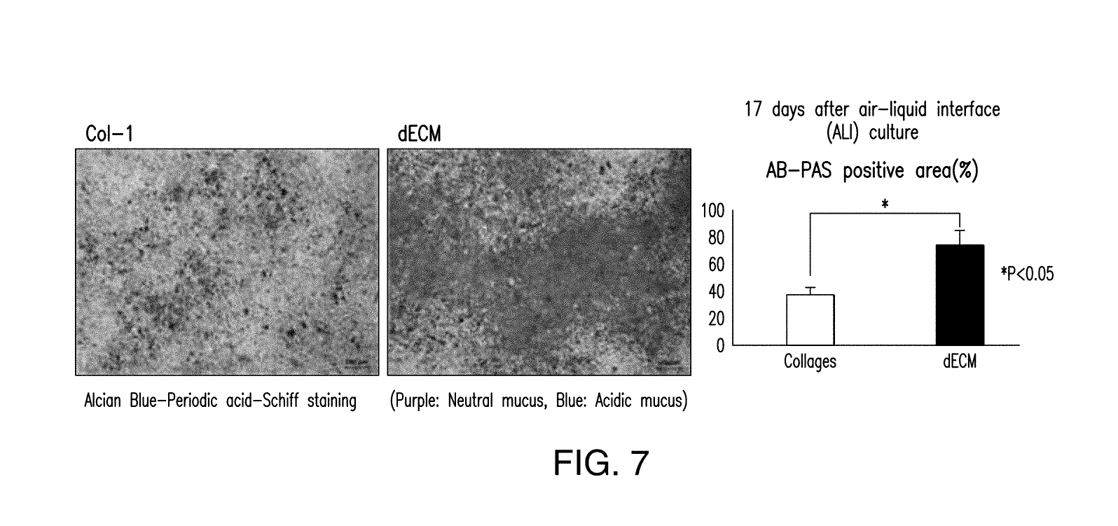

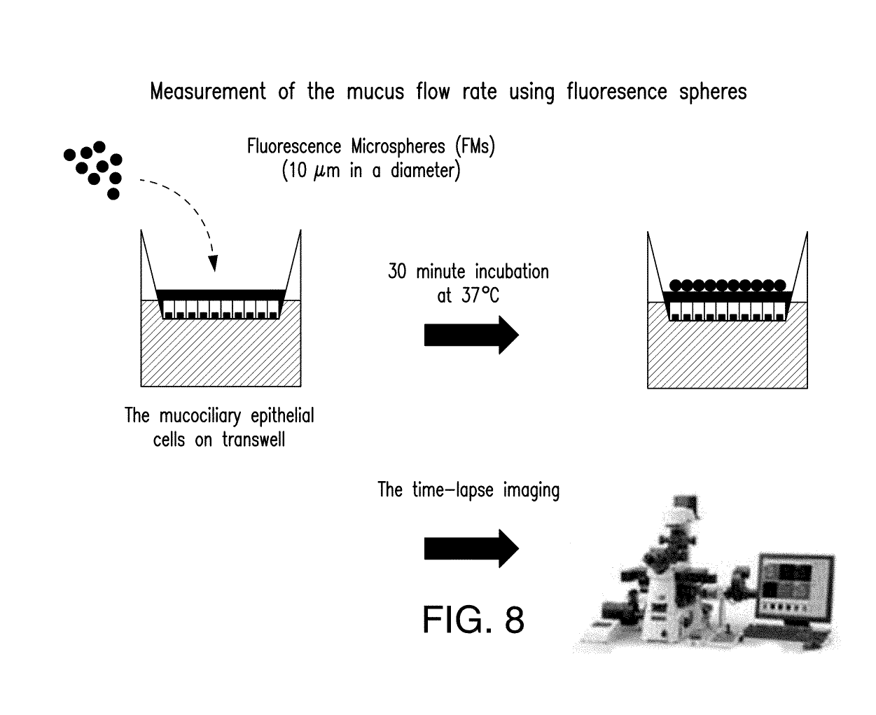

In addition, goblet cell formation, which is another major epithelial cell type in human adult airway, was analyzed by quantifying mucus secretion from the goblet cells were observed using Alcian Blue-Periodic acid-Schiff (AP-PAS). The positively stained area was significantly wider on the dECM hydrogel than that of the collagen hydrogel (FIG. 7).

(3-3) Evaluation of Mucus Flow by the Beating Ciliated Cells

Directional mucus flow, which is an index of the functional tracheal epithelium, was evaluated using 10 μm fluorescent microspheres (FMs). FMs were put on the apical epithelial side of ALI-culture, and transport of the FMs along the surface was recorded by time-lapse imaging using a fluorescence microscope.

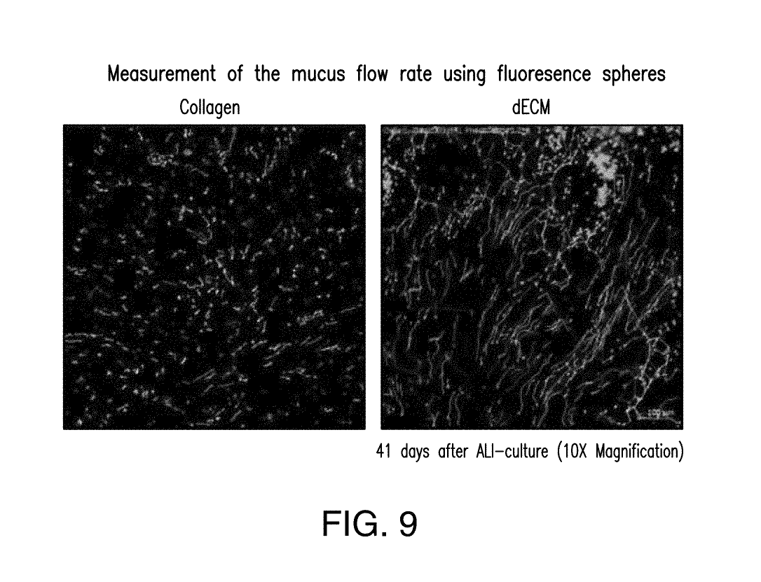

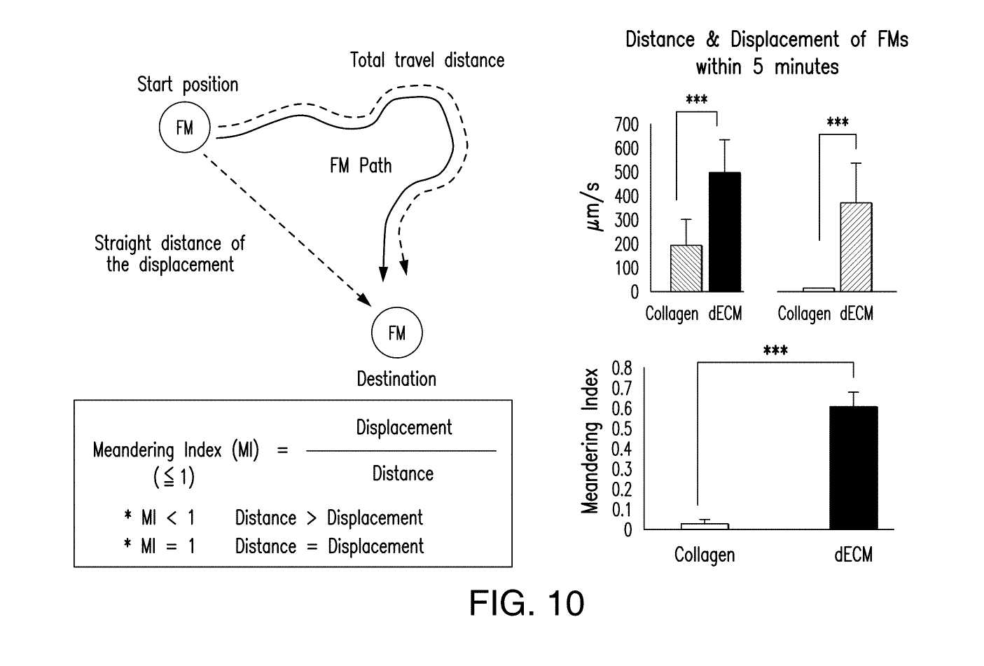

Tracking of individual FMs and calculation of the transport velocity were analyzed with a multi tracking tool in ImageJ (http://imagej.nih.gov/ij/). On the dECM hydrogel, the directional flow of the FMs was formed, while the FMs showed just the beating motions without the mucus flow formation on the collagen hydrogel (FIG. 9). Without being limited to a particular theory, this result indicates that the differentiated ciliated cells on the dECM hydrogel formed the synchronized beating motion of ciliated cells that can be induced by the functional tracheal epithelium. To analyze the speed and directionality of the FMs movement, the trajectories of the FMs movement were translated into the coordinates. The FMs movement on the dECM hydrogel showed high speed and directionality, while the FMs movement on the collagen hydrogel showed low speed and non-directionality (FIG. 10).

### Example 2: The Fabrication and Evaluation of the Vitrified Membrane Comprising the dECM Derived from the Mucosal Tissue of Trachea

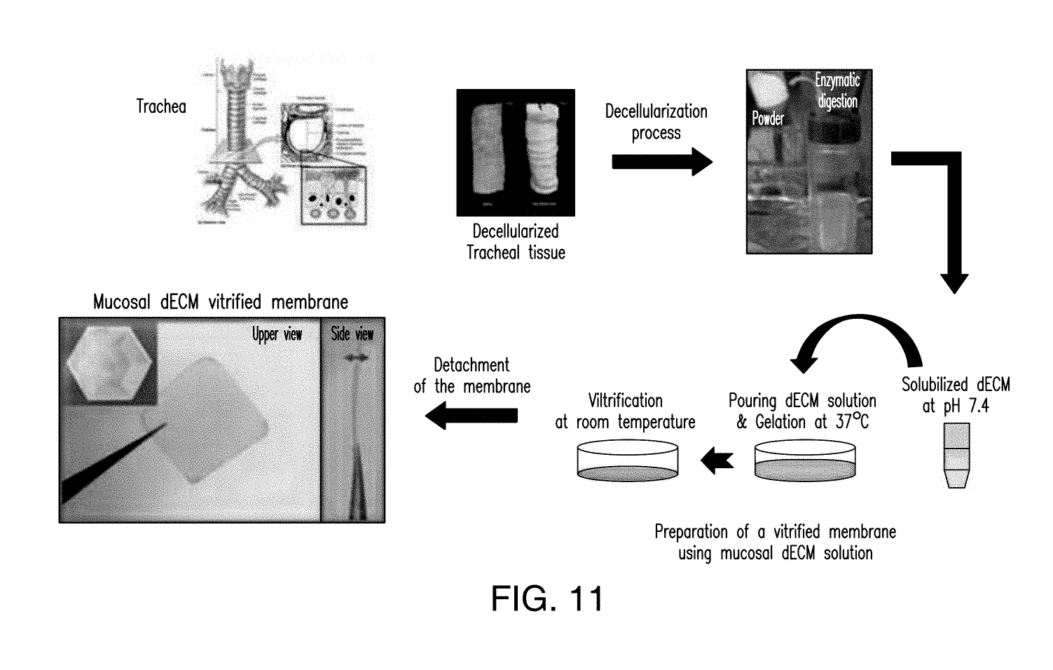

The vitrified membrane comprising the dECM derived from mucosal tissue of porcine trachea were fabricated, and their effects on the cell viability and proliferation were evaluated. These processes are represented as a schematic diagram in FIG. 11.

(1) The Fabrication of the dECM Vitrified Membrane

The dECM pre-gel, which was fabricated at (1) in Example 1, was placed on the surface of the hydrophobic polystyrene film. The non-transparent and soft hydrogel was formed from the pre-gel above by incubation at about 37° C. Then, the thin and transparent vitrified membrane was formed from the hydrogel above by drying at RT (about 25° C.) for 24 hours. Finally, the vitrified membrane was taken off from the styrene film.

(2) Evaluation of the dECM Vitrified Membrane

(2-1) Cell Viability and Proliferation Test

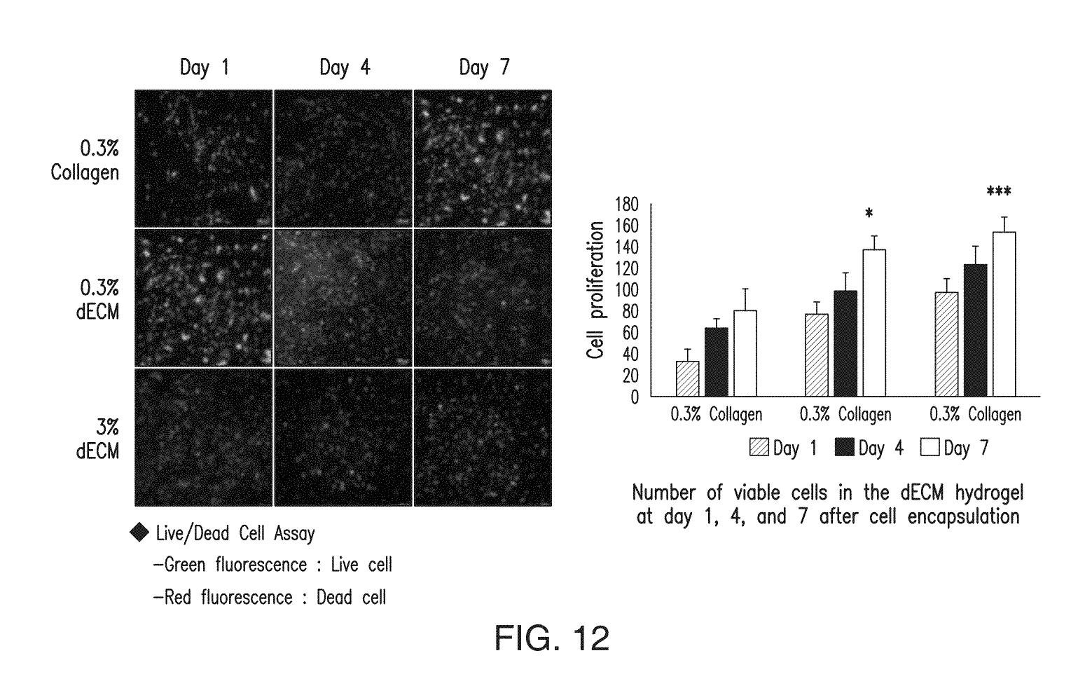

Except the cells type (Embryonic fibroblast; NIH/3T3 cell, ATCC CRL-1658) and the drying process of the hydrogel for 24 hours at RT, all of the process was the same with (3-1) in embodiment (1). Live/Dead assay was conducted at 1, 4, and 7 d, and cell viability and proliferation were significantly higher on the dECM VM than that of collagen (FIG. 12).

(2-2) ALI-Culture Using the dECM Vitrified Membrane

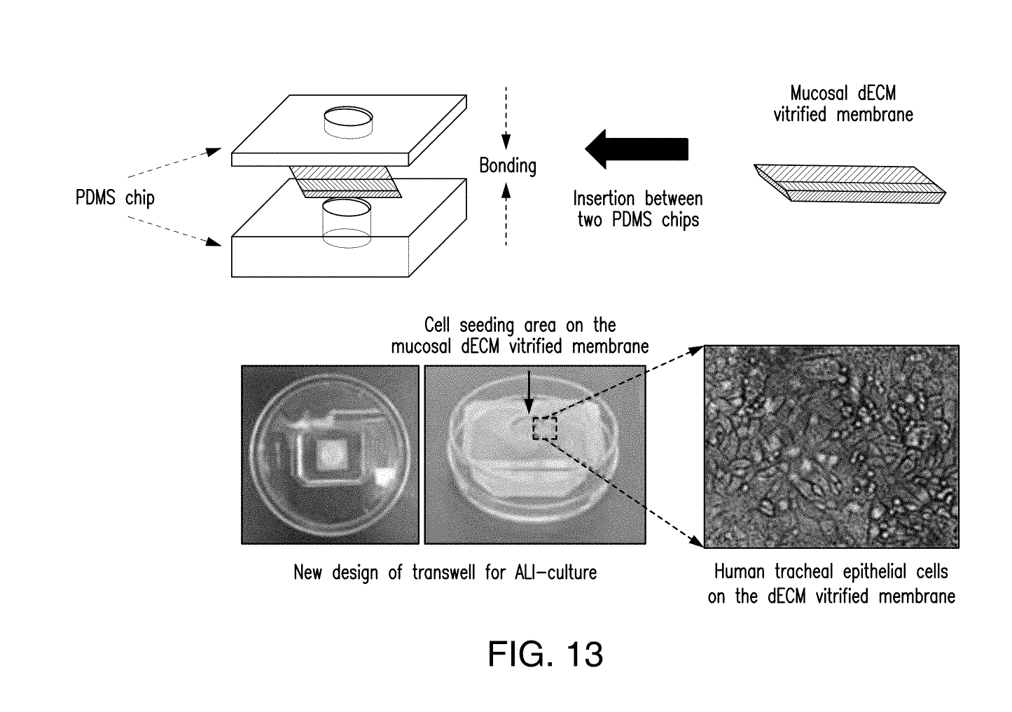

To apply the dECM vitrified membrane to ALI-culture of tracheal epithelial cells, the dECM vitrified membrane fabricated from (3-1) of Example 1 was inserted between the poly(dimethylsiloxane) (PDMS) chip to make a transwell (FIG. 13). hTEpCs were seeded onto the apical part of the dECM vitrified membrane in the transwell, and cultured. Cells proliferated well and rapidly formed tight monolayers on the dECM vitrified membrane of the transwell. This result revealed that the nutrients of the cell culture medium in the bottom-well were delivered to the cells through the dECM vitrified membrane (FIG. 13).

### Example 3: Decellularized Organ-Derived Tissue Engineering Scaffold

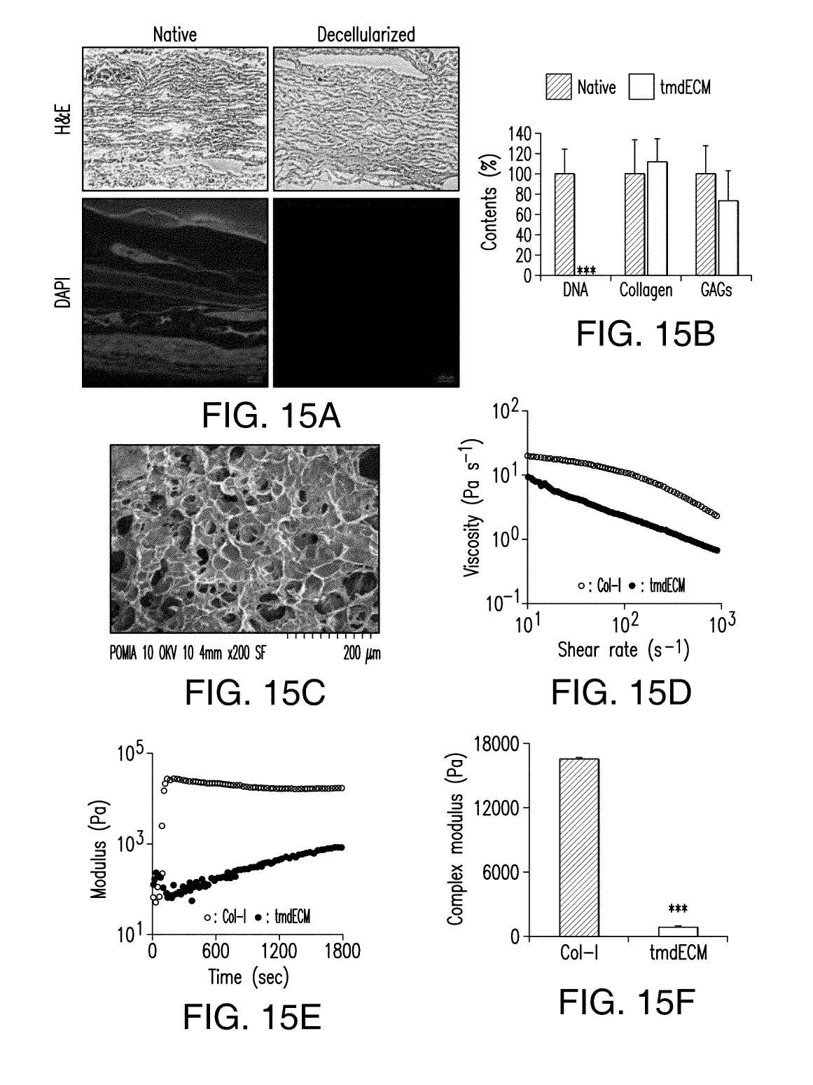

Hematoxylin and eosin (H&E) and DAPI staining were conducted to confirm the absence of cells and cell debris inside the matrix after the decellularization process (FIG. 15A). The removal of cellular components was also evaluated by measuring DNA contents in decellularized mucosal tissue. A 98% reduction in the cellular components (407.86±99.45 ng per mg of native tissue, tmdECM: 10.08±0.103 ng of per mg of tmdECM) was observed. The ECM components including collagen (Col) and glycosaminoglycans (GAGs) were also assessed after decellularization. As shown in FIG. 15B, the Col content increased slightly, while the GAGs content reduced moderately. These data show that the tracheal mucosal tissue was decellularized effectively.

The surface morphology of the freeze-dried tmdECM hydrogel was analyzed by scanning electron microscopy. As shown in FIG. 15C, the surface morphology of the freeze-dried tmdECM hydrogel was highly fibrous and porous.

Rheological properties of the pH adjusted 3% (w/v) tmdECM pre-gel were measured compared to 3% (w/v) Col-1 pre-gel to evaluate their flowability at temperatures below 15° C. As shown in FIG. 15D, the two pre-gels showed shear thinning behavior in the measured shear rate range, and the viscosities of the pre-gels at 10 s⁻¹ shear rate were 22.64 for Col-1 and 10.25 Pa/s for tmdECM when measured at 15° C. Evaluation of the gelation kinetics of the Col-1 and tmdECM pre-gels were analyzed at variable temperatures ranging from 4 to 37° C. with a temperature ramp of 5° C./min. The complex modulus of Col-1 pre-gel increased dramatically by the temperature ramp before reaching 15° C. and then remained constant thereafter. Meanwhile, the complex modulus of tmdECM pre-gel gradually increased by the temperature ramp and even after reaching 37° C. up to 30 min incubation, and then remained constant thereafter (FIG. 15E). This observation indicates that Col-1 pre-gel started gelation immediately by the increasing temperature beyond 4° C., while the tmdECM pre-gel slowly formed a crosslinked gel by incubation at 37° C. for 30 min. The complex modulus of Col-1 and tmdECM gel was compared after incubating the pre-gels at 37° C. for 30 min and Col-1 gel (16.6 kPa) exhibited greater complex modulus than tmdECM gel (0.83 kPa) (FIG. 15F). Without being limited to a particular theory, these data suggest that Col-1 gel can retain its shape more strongly compared to tmdECM gel after gelation. However, tmdECM gel (3% (w/v)) rather exhibited more similar complex modulus to the native lung (Young's modulus: around 1.6 kPa) than that of Col-1 gel.

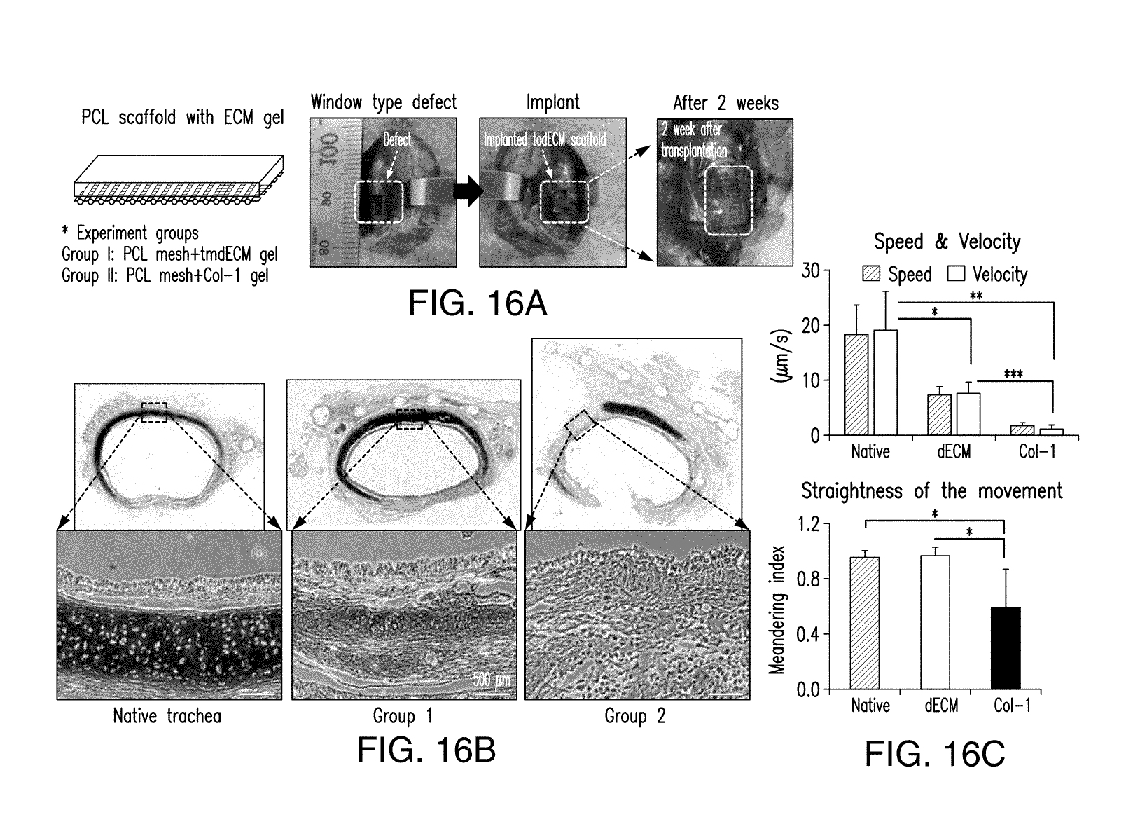

The effect of tmdECM in vivo using a tracheal defect model of a rat compared to Col-1 was also tested. For the fabrication of tracheal grafts, PCL framework was fabricated using 3D printer as a type of thin membrane to load and deliver the tmdECM hydrogel stably to the defect site and the hydrogel surface of the graft faced towards the luminal side of the trachea so that the material can reach directly to the defect site (FIG. 16A).

Histological analysis at 2 weeks post operation showed no significant inflammatory response at the implanted in the tmdECM group while dehiscence occurred by some inflammatory infiltrates between defect and graft in Col-1 group. H&E staining revealed the complete regeneration of defected tracheal wall including tracheal cartilage and mucosal epithelium on the luminal surface in tmdECM group. As shown in FIG. 16B, the thickness and the morphology of the regenerated epithelium of the tmdECM group were nearly identical to those of the native tracheal epithelium. Meanwhile, only a thin, immature epithelium was observed on the luminal surface of the graft in Col-1 group (FIG. 16B).

Mucociliary clearance function was also measured by microscopic analysis using live tracheal tissue specimen on the defect site right after harvesting trachea from the animal at 2 weeks post operation. The velocity of microbeads on the tmdECM group (around 8 μm/sec) was significantly higher than that on the Col-1 group (around 1 μm/sec), and the meandering index represented almost 1 which means the purposive movement of the microbeads on the regenerated tracheal epithelium of tmdECM group, while the value on Col-1 was only 0.6 (FIG. 16C).

Various references are cited herein, the contents of which are hereby incorporated by reference in their entireties.

1. A composition for tissue regeneration comprising decellularized extracellular matrix (dECM) derived from a tissue, wherein the dECM is in the form of at least one of a pre-gel, a hydrogel, and a vitrified membrane.

2. The composition of claim 1, wherein the tissue is selected from the group consisting of skin, eye, heart, liver, intestine, stomach, placenta, cervix, brain, mucosal trachea tissue, and bone.

3. A composition for tracheal mucosal tissue regeneration comprising decellularized extracellular matrix (dECM) derived from tracheal mucosal tissue, wherein the dECM is in the form of at least one of a pre-gel, a hydrogel, and a vitrified membrane.

4. The composition of claim 3, wherein the dECM is derived from mucosal tissue of a porcine trachea.

5. The composition of claim 3, wherein the dECM is derived from mucosal tissue of a human trachea.

6. The composition of claim 1, wherein the dECM is obtained by treatment with one or more detergents selected from the group consisting of sodium dodecyl sulfate, polyethylene glycol p-(1,1,3,3-tetra methyl butyl)-phenyl ether (Triton X-100) and a combination thereof.

7. The composition of claim 6, wherein the dECM is obtained by an additional process of lyophilization and/or pulverization after treatment with the one or more detergents.

8. The composition of claim 1, wherein the dECM pre-gel is obtained by a method comprising: (a) treatment of a dECM derived from tracheal mucosal tissue with a proteolytic enzyme in an acidic solution; and (b) neutralizing the acidic solution by addition of a base.

9. The composition of claim 8, wherein the dECM derived from tracheal mucosal tissue is added to the acidic solution at a concentration between 0.3 and 4 w/v %.

10. The composition of claim 8, wherein the acidic solution comprises an acid selected from the group consisting of acetic acid, hydrochloric acid and a combination thereof.

11. The composition of claim 8, wherein the proteolytic enzyme is selected from the group consisting of pepsin, matrix metalloproteinase and a combination thereof.

12. The composition of claim 1, wherein the dECM pre-gel has a viscosity in a range of about 200 to about 400 Pa·S at a shear rate of 1 s⁻¹.

13. The composition of claim 1, wherein the dECM hydrogel is obtained by gelling a dECM pre-gel at about 37° C.

14. The composition of claim 1, wherein the dECM hydrogel has a membrane form having a thickness of about 200 to about 2000 μm.

15. The composition of claim 1, wherein the dECM vitrified membrane is obtained by drying a dECM hydrogel.

16. The composition of claim 1, wherein the dECM vitrified membrane has a film form having a thickness of about 30 to about 100 μm.

17. A method for generating a decellularized extracellular matrix (dECM) pre-gel composition comprising:
a. treating a tissue with one or more detergents;
b. lyophilizing and/or pulverizing the detergent-treated tissue to generate dECM;
c. treating the dECM with a proteolytic enzyme in an acidic solution; and
d. neutralizing the acidic solution to obtain the dECM pre-gel composition.

18. A method for generating a decellularized extracellular matrix (dECM) hydrogel composition comprising:
a. treating a tissue with one or more detergents;
b. lyophilizing and/or pulverizing the detergent-treated tissue to generate dECM;
c. treating the dECM with a proteolytic enzyme in an acidic solution;
d. neutralizing the acidic solution to obtain a dECM pre-gel composition; and
e. gelling the dECM pre-gel composition to obtain the dECM hydrogel composition.

19. A method for generating a decellularized extracellular matrix (dECM) vitrified membrane comprising:
a. treating a tissue with one or more detergents;
b. lyophilizing and/or pulverizing the detergent-treated tissue to generate dECM;
c. treating the dECM with a proteolytic enzyme in an acidic solution;
d. neutralizing the acidic solution to obtain a dECM pre-gel composition;
e. gelling the dECM pre-gel composition to obtain a dECM hydrogel composition; and
f. drying the dECM hydrogel composition to obtain the dECM vitrified membrane composition.

20. The method of claim 17, wherein the tissue is selected from the group consisting of skin, eye, heart, liver, intestine, stomach, placenta, cervix, brain, mucosal trachea tissue, and bone.

21. The method of claim 17, wherein the tissue is a mucosal trachea tissue.

22. The method of claim 17, wherein the one or more detergents selected from the group consisting of sodium dodecyl sulfate, polyethylene glycol p-(1,1,3,3-tetra methyl butyl)-phenyl ether (Triton X-100) and a combination thereof.

23. The method of claim 17, wherein the proteolytic enzyme is selected from the group consisting of pepsin, matrix metalloproteinase and a combination thereof.

24. The method of claim 17, wherein the acidic solution comprises an acid selected from the group consisting of acetic acid, hydrochloric acid and a combination thereof.

25. The method of claim 17, wherein the dECM is added to the acidic solution at a concentration between 0.3 and 4 w/v %.

26. A method of tissue regeneration or repair comprising administering the composition of claim 1 to a patient.

27. A method of tracheal mucosal tissue regeneration comprising administering the composition for tracheal mucosal tissue regeneration of claim 2 to a patient.

28. The method of claim 27, wherein the composition is injected into a tracheal mucosal tissue injury of the patient.

29. The method of claim 27, wherein the composition is grafted onto a tracheal mucosal tissue injury of the patient.

30. A decellularized extracellular matrix (dECM) composition for analyzing tracheal mucosal tissue in vitro comprising dECM derived from tracheal mucosal tissue, wherein the dECM is in the form of a hydrogel or a vitrified membrane.

31. The composition of claim 30, wherein the dECM hydrogel has a membrane form having a thickness of about 200 to about 2000 μm.

32. The composition of claim 30, wherein the dECM vitrified membrane has a film form having a thickness of about 30 to about 100 μm.

33. A decellularized extracellular matrix (dECM) composition for culturing cells in vitro comprising dECM derived from a tissue, wherein the dECM is in the form of a hydrogel or a vitrified membrane.

34. The composition of claim 33, wherein the dECM hydrogel has a membrane form having a thickness of about 200 to about 2000 μm.

35. The composition of claim 33, wherein the dECM vitrified membrane has a film form having a thickness of about 30 to about 100 μm.

36. The composition of claim 33, wherein the tissue is selected from the group consisting of skin, eye, heart, liver, intestine, stomach, placenta, cervix, brain, mucosal trachea tissue, and bone.
